(19) United States
(12) Patent Application Publication
(10) Pub. No.: US 2025/0081909 A1
(43) Pub. Date: Mar. 13, 2025

(54) METHOD FOR BREEDING IMPROVED VARIETY OF FORAGE SORGHUM-BACILLUS MEGATERIUM SYMBIONT

(71) Applicant: JIANGSU ACADEMY OF AGRICULTURAL SCIENCES, Nanjing, Jiangsu (CN)

(72) Inventors: Xiaoxian ZHONG, Nanjing (CN); Zhengfeng MAO, Nanjing (CN); Wentao XUE, Nanjing (CN); Zhiwei LIU, Nanjing (CN); Chen QIAN, Nanjing (CN); Juanzi WU, Nanjing (CN)

(73) Assignee: JIANGSU ACADEMY OF AGRICULTURAL SCIENCES, Nanjing, Jiangsu (CN)

(21) Appl. No.: 18/730,543

(22) PCT Filed: Oct. 16, 2023
(86) PCT No.: PCT/CN2023/124650

§ 371 Date: Jul. 19, 2024

(30) Foreign Application Priority Data
Oct. 19, 2022 (CN) 202211281389.2

(51) Int. Cl. A01H 1/02; A01G 7/06; A01N 63/22; C12Q 1/689

(52) U.S. Cl. CPC A01H 1/02; A01G 7/06; A01N 63/22; C12Q 1/689

(57) ABSTRACT

A method for breeding an improved variety of a forage sorghum-Bacillus megaterium symbiont, and a CAPS marker for rapidly identifying Bacillus megaterium BM18-2. The method includes: when a stigma of a female parent is exerted for 2-3 d, spraying a Bacillus megaterium solution to the stigma, then pollinating same with pollens from anthers of a male parent after the anthers are exerted for 2-5 d, and harvesting mature seeds. The CAPS marker mainly includes a primer pair designed for an SNP site and endonuclease BspT104I, and upstream and downstream primers are respectively SEQ ID Nos: 1-2. The first method for breeding improved variety of forage sorghum containing endophytic Bacillus megaterium. Produced seeds can significantly increase biomass, crude protein, soluble sugar content and roughage grading index (GI) of plants grown in soil with a salt content ≤8‰; and the CAPS marker can realize rapid qualitative identification of Bacillus megaterium BM18-2.

### TECHNICAL FIELD

The present invention specifically relates to a method for breeding an improved variety of a forage sorghum-Bacillus megaterium symbiont and belongs to the cross technical field of plant-microorganism.

### BACKGROUND

Sorghum forage grows fast in a seedling stage, has good feeding quality, and is warm-season type gramineous forage widely planted at home and abroad. In 1987-2020, a total of 28 of Sorghum sudanense, sorghum, sorghum-Sorghum sudanense hybrids, and Sorghum sudanense-Sorghum propinquum hybrids examined by national grass varieties in China, wherein 12 varieties are tolerant to saline-alkaline stress, and 3 varieties are strongly tolerant to saline stress, namely, “Tiannong No. 2” sorghum-Sorghum sudanense hybrid, “Tiannong Qingsi No. 1” sorghum-Sorghum sudanense hybrid and “Ningnong” Sorghum sudanense all grow well in soil with a salt (NaCl) content ≤4‰. However, generally, the varieties tolerant to saline-alkaline stress are relatively few, and the breeding method needs to be improved.

The existing research shows that the salt tolerance is quantitative character, controlled by polygenes and related to the morphological adaptation and biochemical adaptation of plants. Therefore, it is very difficult to obtain a saline-tolerant plant material through conventional breeding and gene transformation. In recent years, the thought of plant breeding has been changed fundamentally. A breeding strategy of “using plants as a holobiont, an ecological and evolutionary unit of a host and microbial flora” has become the leading edge of research at home and abroad (Vandenkoornhuyse, P. et al. The importance of the Microbiome of the Plant Holobiont, New Phytologist, 2015, 206(4), 1196-1206; Zhong Wei, Alexandre Jousset. Plant Breeding Goes Microbia, Trend in Plant Scienc, 2017, 22(7): 555-558). Endophytic fungal symbionts are hot spots of research at home and abroad, but formal reports of endophytic bacterial symbiont breeding are few. Only the Austria Birgit Mitter team introduces specific endophytes through flowers to obtain chili, soybean, corn and wheat symbiont F1 generation seeds containing endophytic bacteria P. phytofirmans PsJN (Mitter B et al. A New Approach to Modify Plant Microbiomes and Traits by Introducing Beneficial Bacteria at Flowering into Progeny Seeds. Frontiers in Microbilogy, 2017, 8:11). These plants belong to grain crops and are greatly different from sorghum hybrids.

Bacillus megaterium is an important bacterium of the genus Bacillus, widely exists in nature, and is friendly to human, livestock and environment. The existing research shows that used as a microbial fertilizer, the Bacillus megaterium endophytic in plants can promote the growth of the plants by fixing nitrogen, dissolving phosphorus, promoting growth hormones, etc. to improve the tolerance of the host plants to salt stress (Hu Xiaojia and Jiang Mulan, Effect of Bacillus megaterium (A6) in Promoting Rape in Red-yellow Soil, Chinese Journal of Oil Crop Sciences, 2003(04):107-108; Luo Huan, Wu Huijun, Xie Yongli et al. Effects of Bacillus megaterium CJLC2 on Growth of Tomatoes and Saline-tolerant Physiological and Biochemical Indicators under Salt Stress, Journal of Plant Protection, 2013, 40(05): 431-436; Li Xia, Geng Xiaoyan, Xie Rongrong et al. The endophytic Bacteria Isolated from Elephant Grass (Pennisetum purpureum Schumach) Promote Plant Growth and Enhance Salt Tolerance of Hybrid Pennisetum. Biotechnology for Biofuels, 2016, 9:190:1-12). However, the external application effect of the existing Bacillus megaterium is greatly affected by the environment, and the standard operation requirement on an operator is relatively high. In 2018, Zhong Xiaoxian et al. disclosed a cadmium-enriching and growth-promoting hybrid Pennisetum alopecuroides endophytic Bacillus megaterium BM18-2 (Patent number: ZL201810143961.6).

Breeding of an improved variety of an endophytic Bacillus megaterium symbiont in gramineous forage and use thereof in saline soil improvement are not reported at home and abroad.

### SUMMARY

Aiming at the shortcomings of the prior art, a first objective of the present invention is to provide a method for breeding an improved variety of a forage sorghum-Bacillus megaterium symbiont and evaluate the grass yield and the feeding quality of the bacterial grass symbiont under different salt stresses so as to provide a new strategy for saline soil improvement.

A second objective of the present invention is to provide seeds obtained by the method for breeding an improved variety of a forage sorghum-Bacillus megaterium symbiont mentioned above.

A third objective of the present invention is to provide use of the method for breeding an improved variety of a forage sorghum-Bacillus megaterium symbiont mentioned above in saline soil improvement.

A fourth objective of the present invention is to provide use of Bacillus megaterium, or a microbial agent containing the Bacillus megaterium, or a culture containing the Bacillus megaterium, or a microbial fertilizer containing the Bacillus megaterium in breeding an endophytic bacterial symbiont.

A fifth objective of the present invention is to provide a CAPS marker for rapidly identifying Bacillus megaterium BM18-2.

The objectives of the present invention can be realized by the following technical solutions:

On a first aspect, the present invention sets forth a method for breeding an improved variety of a forage sorghum-Bacillus megaterium symbiont, comprising the following steps: when a stigma of a female parent is exerted for 2-3 d, spraying a Bacillus megaterium solution to the stigma, then pollinating same with pollens from anthers of a male parent after the anthers are exerted for 2-5 d, and harvesting mature seeds.

In some preferred technical solutions of the present application, the Bacillus megaterium is Bacillus megaterium BM18-2.

The Bacillus megaterium BM18-2 with the patent number of ZL201810143961.6 is preserved in China Center for Type Culture Collection addressed in Wuhan University, China on Nov. 10, 2017 with the deposit number of CCTCC NO: M2017679.

Preferably, the Bacillus megaterium BM18-2 used in the present invention is a Bacillus megaterium BM18-2 product with the effective viable count of ≥200 million/mL and is a microbial fertilizer product produced by Yangzhou Luyuan Biochemical Co., Ltd.

When the Bacillus megaterium BM18-2 is used, the Bacillus megaterium BM18-2 solution product with the effective viable count of ≥200 million/mL is diluted for 300-1,000 times to be sprayed on the stigma of the female parent.

In some preferred technical solutions of the present application, the female parent is a Sorghum sudanense-Sorghum propinquum hybrid.

Preferably, the female parent is a tetraploid Sorghum sudanense-Sorghum propinquum hybrid SS2010-2.

In some preferred technical solutions of the present application, the male parent is sweet sorghum.

Preferably, the male parent is sweet sorghum SS2015.

The present invention provides the first method for breeding the improved variety of the perennial forage sorghum containing endophytic Bacillus megaterium. The produced seeds can significantly increase the biomass, crude protein, soluble sugar content and roughage grading index (GI) of plants grown in soil with a salt content ≤8‰.

On a second aspect, the present invention further sets forth seeds obtained by the method for breeding an improved variety of a forage sorghum-Bacillus megaterium symbiont mentioned above.

On a third aspect, the present invention further sets forth use of the method for breeding an improved variety of a forage sorghum-Bacillus megaterium symbiont mentioned above, and the seeds mentioned above in saline soil improvement.

The seeds obtained by the breeding method of the present invention can grow well in soil with a salt (NaCl) content ≤4‰ and also grow well in soil with a salt content ≤8‰, wherein the biomass, crude protein, soluble sugar content and roughage grading index (GI) are all obviously higher than those of a control group, which brings a new thought for saline soil improvement.

On a fourth aspect, the present invention further sets forth use of Bacillus megaterium, or a microbial agent containing the Bacillus megaterium, or a culture containing the Bacillus megaterium, or a microbial fertilizer containing the Bacillus megaterium in breeding an endophytic bacterial symbiont.

Preferably, the Bacillus megaterium is Bacillus megaterium BM18-2.

Preferably, breeding the endophytic bacterial symbiont is breeding an improved variety of a forage sorghum-Bacillus megaterium symbiont.

Specifically, the present invention sets forth use of the Bacillus megaterium BM18-2, or the microbial agent containing the Bacillus megaterium BM18-2, or the culture containing the Bacillus megaterium BM18-2, or the microbial fertilizer containing the Bacillus megaterium BM18-2 in breeding the improved variety of a forage sorghum-Bacillus megaterium symbiont.

On a fifth aspect, the present invention further sets forth a CAPS marker for rapidly identifying Bacillus megaterium BM18-2, mainly comprising a primer pair designed for an SNP site and endonuclease BspT104I, wherein
an upstream primer and a downstream primer of the primer pair are respectively as follows:
the upstream primer F: GTATTACTTGAAGGCAATCGTCCAGC shown in SEQ ID No: 1; and
the downstream primer R: AGCGTCTTCAGCAATGATGACTTCC shown in SEQ ID No: 2.

During PCR amplification, an amplified product contains a TTCGAA restriction enzyme cutting site of restriction endonuclease BspT104I.

The present invention further sets forth use of the CAPS marker mentioned above in any one of the following (A1)-(A4):
(A1) identifying Bacillus megaterium BM18-2;
(A2) preparing a kit for detecting the Bacillus megaterium BM18-2;
(A3) determining whether a sample to be detected contains the Bacillus megaterium BM18-2; and
(A4) assisting in breeding an endophytic bacterial symbiont.

The breeding an endophytic bacterial symbiont is breeding an improved variety of a crop-Bacillus megaterium symbiont.

The crop is an agricultural crop, forage, etc.; the agricultural crop is corn, soybeans, rice, etc.; the forage is forage sorghum, etc.; and the Bacillus megaterium is Bacillus megaterium BM18-2.

Specifically, when use of the Bacillus megaterium BM18-2, or the microbial agent containing the Bacillus megaterium BM18-2, or the culture containing the Bacillus megaterium BM18-2, or the microbial fertilizer containing the Bacillus megaterium BM18-2 in breeding the improved variety of a forage sorghum-Bacillus megaterium symbiont is studied, the Bacillus megaterium BM18-2 can be identified using the CAPS marker mentioned above.

As a preferred technical solution of the present application, identifying the Bacillus megaterium BM18-2 comprises the following specific steps:
(1) performing PCR amplification using the primer pair mentioned above to obtain a PCR amplified product;
(2) performing enzyme digestion on the PCR amplified product using the restriction endonuclease BspT104I mentioned above to obtain an enzyme-digested product; and
(3) performing electrophoresis on the enzyme-digested product and determining whether the enzyme-digested product contains the Bacillus megaterium BM18-2 or not according to the type of a band obtained by the electrophoresis.

The present invention further sets forth a kit for rapidly identifying Bacillus megaterium BM18-2, containing the CAPS marker mentioned above.

### Beneficial Effects

The present invention provides the first method for breeding the improved variety of the perennial forage sorghum containing endophytic Bacillus megaterium. The produced seeds can significantly increase the biomass, crude protein, soluble sugar content and roughage grading index (GI) of plants grown in soil with a salt content ≤8‰.

The present invention provides a CAPS marker for rapidly identifying Bacillus megaterium BM18-2. The marked restriction enzyme cutting site is found only in the Bacillus megaterium BM18-2 by NCBI database search.

### BRIEF DESCRIPTION OF THE DRAWINGS

FIG. 1 shows the process of breeding a field improved variety perennial forage sorghum-Bacillus megaterium symbiont, wherein A is a tetraploid sterile line Sorghum sudanense-Sorghum propinquum hybrid SS2010-2 (female parent); and B is a diploid sweet sorghum SS2015 (male parent);

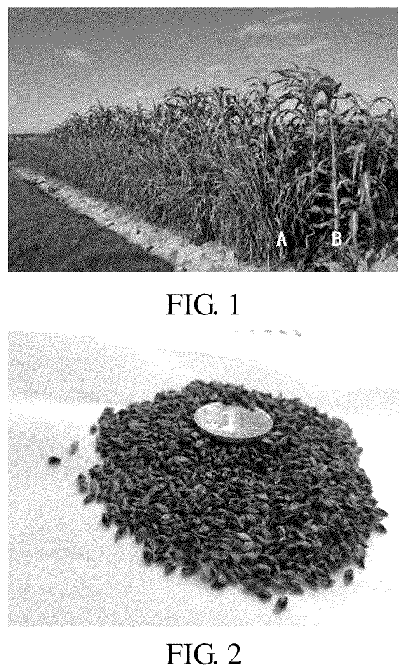

FIG. 2 shows seeds of the perennial forage sorghum-Bacillus megaterium symbiont;

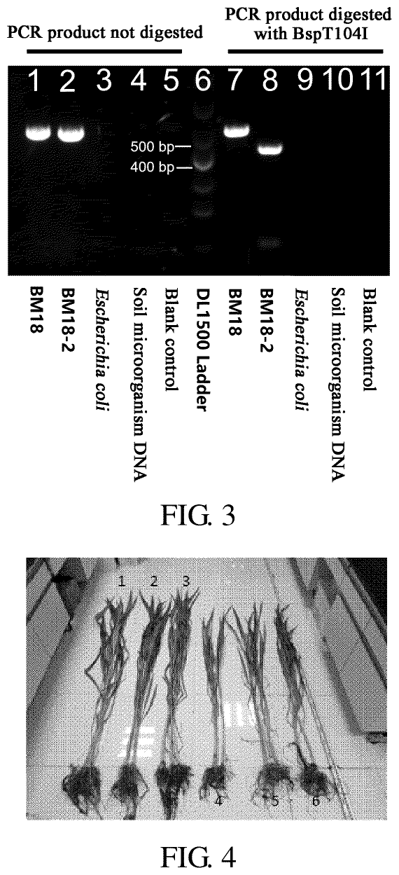

FIG. 3 shows a 3% agarose electrophoresis of Bacillus megaterium BM18-2,
wherein, lanes 1-5 are specific primer PCR products not digested with restriction endonuclease, lanes 7-11 are specific primer PCR products digested with restriction endonuclease BspT104I, lane 6 is DL1500 μladder (TAKARA), the DNA in lanes 1 and 7 belongs to strain BM18, the DNA in lanes 2 and 8 belongs to strain BM18-2, the DNA in lanes 3 and 9 belongs to Escherichia coli, the DNA in lanes 4 and 10 belongs to a soil microorganism, and the DNA in lanes 5 and 11 belongs to a blank control; and
as shown in FIG. 3, in the PCR product of the DNA sample not digested, the strains BM18 and BM18-2 both contain 521-bp characteristic bands, but no amplification exists in other bacterial DNA samples; and in the products digested with the restriction endonuclease BspT104I, only the strain BM18-2 contains the 418-bp characteristic band, but the strain BM18 still contains the 521-bp characteristic band; and

FIG. 4 shows effects of salt (8 g/kg NaCl) stress on growth of the forage sorghum-Bacillus megaterium symbiont and a control thereof, wherein 1-3: forage sorghum-Bacillus megaterium symbiont; and 4-6: forage sorghum.

### DETAILED DESCRIPTION

The present invention is further described in detail below with reference to example. All of the used reagents or instruments which are not specified with manufacturers are conventional commercially-available products.

### Example 1

### (1) Selection of Forage Sorghum Parents

A tetraploid sterile line Sorghum sudanense-Sorghum propinquum hybrid SS2010-2 (female parent): obtained by doubling somatic chromosomes of an F1 generation hybrid of a diploid annual Sorghum sudanense 2098 and perennial Sorghum propinquum by colchicine induction (common general knowledge, Cui Lili, Study on Structural Characteristics and Energy Utilization Potential of Tetraploid Sorghum Sudanense New Germplasm, Master Academic Paper of Nanjing Agriculture University, 2013), and perennial; and sweet sorghum SS2015 (male parent): a farmer seed single spike was collected by a researcher Gu Hongru of Institute of Animal Husbandry, Jiangsu Academy of Agricultural Sciences from Xindian town, Rudong county, Jiangsu province in 2015, and numbered SS2015, the collected single spike seeds were subjected to field propagation in 2016-2019, and the plant was annual.

### (2) Production of Bacillus Megaterium BM18-2 Solution

The Bacillus megaterium BM18-2 was granted with the patent (Zhong Xiaoxian, Qian Chen, Liu Zhiwei, Wu Juanzi, Zhang Jianli, and Pan Yumei, Cadmium-enriching and Growth-promoting Hybrid Pennisetum Alopecuroides Endophytic Bacillus Megaterium BM18-2 and Use thereof, Patent number: ZL201810143961.6) and preserved in China Center for Type Culture Collection addressed in Wuhan University, China on Nov. 10, 2017 with the deposit number of CCTCC NO: M2017679, was a microbial fertilizer product produced by Yangzhou Luyuan Biochemical Co., Ltd., and had the effective viable count of ≥200 million/mL.

### (3) Breeding of Improved Variety of Perennial Forage Sorghum-Bacillus Megaterium Symbiont

In early October 2020, when a stigma of the female parent perennial tetraploid Sorghum sudanense-Sorghum propinquum hybrid SS2010-2 was exerted for 2-3 d, the Bacillus megaterium BM18-2 solution product with the effective viable count of ≥200 million/mL was diluted for 300-1,000 times to be sprayed to the stigma, then the female parent was pollinated with pollens from anthers of the male parent annual sweet sorghum SS2015 after the anthers were exerted for 2-5 d, and mature seeds were harvested in early to mid-November 2020.

### (4) Construction of Accurate Identification Method of Bacillus Megaterium BM18-2

BM18-2 was obtained by mutagenizing and separating wild-type BM18 in a high-cadmium stress culture medium. The strain had an SNP nonsynonymous mutation of Q to E in a coding gene RNA polymerase through genome sequencing comparison. High-specificity PCR amplification primers (F: GTATTACTTGAAGGCAATCGTCCAGC shown in SEQ ID No: 1; R: AGCGTCTTCAGCAATGATGACTTCC shown in SEQ ID No: 2) were designed and screened according to the SNP site. The primers had 521-bp fragment amplification in the wild-type BM18 and mutant strain BM18-2 of the Bacillus megaterium and had no amplification in other bacteria. Due to the single nucleotide mutation of the DNA from C to G, only the PCR amplified fragment of BM18-2 contained a TTCGAA recognition site which can be digested by restriction endonuclease BspT104I, while TTCCAA in the primer PCR amplified fragment of the wild-type BM18 cannot be correspondingly recognized and digested. Finally, the 418-bp enzyme-digested fragment existed only after the enzyme digestion of the primer amplified product of the strain BM18-2, such that the strain BM18-2 can be accurately identified.

### (5) Detection of Perennial Forage Sorghum-Bacillus Megaterium Symbiont Seed BM18-2

After the surfaces of the seeds of the bred improved variety harvested in (3) were disinfected, the single seeds were ground under the aseptic condition, inoculated into an LB culture medium and cultured in a 30° C. incubator for 72-96 h. The bacterial plaques in a culture dish were picked using a 10-1 pipette tip under the aseptic condition and mixed in 18 μl of a PCR reaction solution. The reaction solution system was: 8.6 μl of 2×Rapid Taq Master Mix (P222-AA, Vazyme), 0.4 μl of 10 m F primer, 0.4 μl of 10 m R primer, and 8.6 μl of ddH₂O, wherein the F primer was GTATTACTTGAAGGCAATCGTCCAGC and the R primer was AGCGTCTTCAGCAATGATGACTTCC. The PCR program of the bacterial solution was: 95° C. for 3 min, 35 cycles of 95° C. for 15 sec, 60° C. for 15 sec, and 72° C. for 15 sec, annealing at 72° C. for 5 min. The enzyme digestion reaction on the PCR product was: the PCR product was divided into two parts, one of which was added with endonuclease BspT104I (1225A, TAKARA) and the reaction system not added with the enzyme was used as a control, reaction was performed at 37° C. for 30 min, the enzyme digestion system was 8 μl of the PCR product of the bacterial solution, 1 μl of 10×L buffer and 1 μl of endonuclease BspT104I, and the control system was 8 μl of the PCR product of the bacterial solution, 1 μl of 10×L buffer and 1 μl of ddH₂O. The electrophoresis identification of the enzyme-digested products: the enzyme-digested products and the control were subjected to electrophoresis using 3% agarose, and a 418-bp specific characteristic band can be seen in the strain BM18-2.

### (6) Evaluation on Salt Tolerance of Perennial Forage Sorghum-Bacillus Megaterium Symbiont

In 2021, in a glass greenhouse of a national forage breeding innovation base of the Jiangsu Academy of Agricultural Sciences, located at 118° 57′ of an east longitude and 32° 03′ of a northern latitude, the forage sorghum-Bacillus megaterium symbiont seeds bred in (3) were sown on April 2 and perennial forage sorghum seeds bred normally were sown as a control at the same time. Symbiont and control seedlings at 3-4 μleaf-stage with relatively consistent growth were respectively selected and transplanted to plastic pots with 3 plants per pot on April 28. The pot body had the diameter of 17 cm and the height of 20 cm, the soil was matrix soil (the main component was white peat with the pH=6.0 and the soil structure was medium thick) and uniformly mixed with chemically pure NaCl at a weight ratio, the NaCl concentrations for the test treatment were 0 g/kg, 4 g/kg, 6 g/kg and 8 g/kg respectively, and each treatment was repeated for 3 times. The aboveground parts of the plants were harvested on June 15. After the stems and leaves were separated, the aboveground parts were subjected to enzyme-deactivation at 105° C. for 30 min, dried at 70° C. to constant weight, and weighed. The dried sample was taken. The content of crude protein (CP) was determined by a Kjeldahl method, the content of crude fat was determined by a Soxhlet extraction method, the content of neutral detergent fiber (NDF) and acid detergent fiber (ADF) was determined by a Van Soets method, and the content of soluble sugar was determined by an anthrone colorimetry. The roughage grading index (GI) was calculated according to the content of CP, NDF and ADF (Zhang Jikun, Lu Dexun, Liu Jianxin et al. Research Status and Progress of Roughage Quality Evaluation Index [J], Pratacultural Science, 2004, 21(9):7): GI (Mcal)=ME(Mcal/kg)×DMI(kg/d)×CP(% DM)/NDF(% DM), wherein ME=4.2014+0.0236×(ADF/10)+0.1794×(CP/10), DMI(% DM)=120/(NDF/10).

The results showed that the dry weight of stems and leaves and the total dry weight of aboveground parts of plants of the forage sorghum-Bacillus megaterium symbiont under the stress of the same concentration of salt were remarkably improved compared with those of the control forage sorghum. The total dry weight of the aboveground parts of the forage sorghum-Bacillus megaterium symbiont plants was improved by 7.73%, 10.01%, 16.34% and 18.24% respectively compared with that of the control with remarkable difference (all P<0.01) under the stress by NaCl at concentrations of 0, 4, 6 and 8 g/kg.

TABLE 1
Weight difference between dry matter of forage sorghum-bacillus megaterium
symbiont and control stressed by NaCl at different concentrations

| | | | | Total dry weight |
|---|---|---|---|---|
| | NaCl | Dry weight | Dry weight | of aboveground |
| Tested | concentration | of stems | of leaves | parts of plants |
| materials | (g/kg) | (g) | (g) | (g) |
| Forage sorghum | 0 | 5.36 ± 0.27 bB | 12.37 ± 0.12 bB | 17.73 ± 0.18 bB |
| | 4 | 4.84 ± 0.24 dD | 10.65 ± 0.11 dD | 15.49 ± 0.15 dD |
| | 6 | 3.60 ± 0.18 gG | 9.01 ± 0.09 gG | 12.61 ± 0.13 gG |
| | 8 | 3.13 ± 0.16 hH | 8.11 ± 0.08 hH | 11.24 ± 0.11 hH |
| Forage | 0 | 5.95 ± 0.30 aA | 13.14 ± 0.13 aA | 19.10 ± 0.19 aA |
| sorghum-bacillus | 4 | 5.21 ± 0.26 cC | 11.83 ± 0.12 cC | 17.04 ± 0.17 cC |
| megaterium | 6 | 4.54 ± 0.23 eE | 10.13 ± 0.10 eE | 14.67 ± 0.15 eE |
| symbiont | 8 | 3.90 ± 0.19 fF | 9.39 ± 0.09 fF | 13.29 ± 0.13 fF |

Note: the lower case letters in the same column indicate significant difference (P<0.05) and the upper case letters indicate remarkably significant difference (P<0.01), similarly hereinafter.

The content of the crude protein and soluble sugar of the forage sorghum-Bacillus megatherium symbiont plants was obviously higher than that of the control forage sorghum (Table 2), wherein when the NaCl concentration was 0, 4, 6 and 8 g/kg, the content of the crude protein of the symbiont was significantly improved by 10.581%, 16.17%, 10.97% and 4.33% respectively compared with that of the control, and the content of the soluble sugar was significantly improved by 17.20%, 13.14%, 17.90% and 13.36% respectively; and when the NaCl concentration was 4 and 6 g/kg, the content of the crude fat was significantly improved by 9.05% and 6.50% respectively.

TABLE 2
Feeding quality differences between forage sorghum-bacillus megaterium
symbiont and control stressed by NaCl at different concentrations

| | NaCl | | | |
|---|---|---|---|---|
| Tested | concentration | Crude protein | Crude fat | Soluble sugar |
| materials | (g/kg) | (%) | (%) | (%) |
| Forage sorghum | 0 | 6.43 ± 0.14fE | 2.33 ± 0.03bB | 10.35 ± 0.49dD |
| | 4 | 6.74 ± 0.14eE | 2.43 ± 0.05bB | 11.95 ± 0.18cC |
| | 6 | 7.29 ± 0.17dCD | 2.00 ± 0.08dCD | 12.12 ± 0.20cC |
| | 8 | 7.62 ± 0.07cBC | 1.94 ± 0.06dD | 12.72 ± 0.40cBC |
| Forage | 0 | 7.11 ± 0.20dD | 2.39 ± 0.08bB | 12.13 ± 0.42cC |
| sorghum-bacillus | 4 | 7.83 ± 0.13bcAB | 2.65 ± 0.20aA | 13.52 ± 0.47bAB |
| megaterium | 6 | 8.09 ± 0.07aA | 2.13 ± 0.12cC | 14.29 ± 0.70abA |
| symbiont | 8 | 7.95 ± 0.16abAB | 1.94 ± 0.04dD | 14.42 ± 0.34aA |

When the NaCl concentration was 0, 6 or 8 g/kg, the differences of the content of the neutral detergent fiber and acid detergent fiber and in-vitro digestibility of the forage sorghum-Bacillus megaterium symbiont plants were not significant. When the NaCl concentration was 4 g/kg, the content of the neutral detergent fiber of the symbiont was significantly higher than that of the control and the content of the acid detergent fiber was significantly lower than that of the control. When the NaCl concentration was 0, 4, 6 and 8 g/kg, the roughage grading index (GI) of the symbiont was respectively improved by 9.56%, 18.04%, 15.49% and 6.62% respectively compared with that of the control with a significant or extremely significant difference (Table 3).

TABLE 3
Feeding quality differences between forage sorghum-bacillus megaterium
symbiont and control stressed by NaCl at different concentrations

| | NaCl | Neutral | Acid detergent | Roughage |
|---|---|---|---|---|
| | concentration | detergent fiber | fiber | grading index |
| Tested materials | (g/kg) | NDF (%) | ADF (%) | (GI) |
| Forage sorghum | 0 | 45.68 ± 2.18dC | 22.08 ± 0.50aA | 7.74 ± 0.14cdBC |
| | 4 | 49.47 ± 0.52cB | 21.15 ± 0.18bAB | 7.04 ± 0.12eD |
| | 6 | 51.22 ± 0.68abAB | 20.24 ± 0.32cBC | 7.36 ± 0.0.30deCD |
| | 8 | 52.73 ± 0.68aA | 19.60 ± 0.69cdCD | 7.40 ± 0.24deCD |
| Forage | 0 | 46.20 ± 1.22dC | 21.78 ± 0.31abA | 8.48 ± 0.49aA |
| sorghum-bacillus | 4 | 51.04 ± 1.01bAB | 20.24 ± 0.32cBC | 8.31 ± 0.11abAB |
| megaterium | 6 | 51.66 ± 0.76abA | 19.60 ± 0.12cdCD | 8.50 ± 0.17aA |
| symbiont | 8 | 52.70 ± 0.49aA | 19.08 ± 0.12dD | 7.89 ± 0.18bcABC |

The protective content of the present invention is not limited to the described example. Changes and advantages that can be easily figured out by a person skilled in the art without departing the spirit and scope of the present invention are included in the present invention and subject to the protection scope of the claims.

1. A method for breeding an improved variety of a forage sorghum-Bacillus megaterium symbiont, comprising the following steps: when a stigma of a female parent is exerted for 2-3 d, spraying a Bacillus megaterium solution to the stigma, then pollinating same with pollens from anthers of a male parent after the anthers are exerted for 2-5 d, and harvesting mature seeds.

2. A method for breeding an improved variety of a forage sorghum-Bacillus megaterium symbiont according to claim 1, wherein the Bacillus megaterium is Bacillus megaterium BM18-2.

3. The method for breeding an improved variety of a forage sorghum-Bacillus megaterium symbiont according to claim 1, wherein the female parent is a Sorghum sudanense-Sorghum propinquum hybrid.

4. The method for breeding an improved variety of a forage sorghum-Bacillus megaterium symbiont according to claim 1, wherein the male parent is sweet sorghum.

5. The seeds obtained by the method for breeding an improved variety of a forage sorghum-Bacillus megaterium symbiont according to claim 1.

6. A method for saline soil improvement comprising performing the method for breeding an improved variety of a forage sorghum-Bacillus megaterium symbiont according to claim 1, and providing the seeds obtained by the method for breeding an improved variety of a forage sorghum-Bacillus megaterium symbiont for saline soil improvement.

7. A method comprising breeding an endophytic bacterial symbiont using Bacillus megaterium, or a microbial agent containing the Bacillus megaterium, or a culture containing the Bacillus megaterium, or a microbial fertilizer containing the Bacillus megaterium.

8. A CAPS marker for rapidly identifying Bacillus megaterium BM18-2, mainly comprising a primer pair designed for an SNP site and endonuclease BspT104I, wherein
an upstream primer and a downstream primer of the primer pair are respectively as follows:
the upstream primer F: GTATTACTTGAAGGCAATCGTCCAGC shown in SEQ ID No: 1; and
the downstream primer R: AGCGTCTTCAGCAATGATGACTTCC shown in SEQ ID No: 2.

9. A method comprising
identifying Bacillus megaterium BM18-2 and/or determining whether a sample to be detected contains the Bacillus megaterium BM18-2 using the CAPS marker according to claim 8.

10. A kit for rapidly identifying Bacillus megaterium BM18-2, containing the CAPS marker according to claim 8.

11. A method comprising:
preparing a kit for detecting Bacillus megaterium BM18-2, the kit including the CAPS marker according to claim 8.

12. A method comprising:
assisting in breeding an endophytic bacterial symbiont using the CAPS marker according to claim 8.


SEQUENCE LISTING (2 sequences)

SEQ ID NO: 1 - DNA, 26 bp, synthetic construct
  source 1..26: mol_type = other DNA
gtattacttgaaggcaatcgtccagc

SEQ ID NO: 2 - DNA, 25 bp, synthetic construct
  source 1..25: mol_type = other DNA
agcgtcttcagcaatgatgacttcc